(19) United States
(12) Patent Application Publication
(10) Pub. No.: US 2026/0132372 A1
(43) Pub. Date: May 14, 2026

(54) METHOD FOR REMOVING UNDIFFERENTIATED CELLS

(71) Applicants: Nanyang Technological University, Singapore (SG); Massachusetts Institute of Technology, Cambridge, MA (US); Singapore-MIT Alliance For Research and Technology, Singapore (SG)

(72) Inventors: Jongyoon HAN, Cambridge, MA (US); Sing Yian CHEW, Singapore (SG); Tan Dai NGUYEN, Singapore (SG); Zu Yao Jerome TAN, Singapore (SG); Hyungkook JEON, Cambridge, MA (US); Daniel Ninio ROXBY, Singapore (SG); Vaibavi SRIRANGAM RAMANUJAM, Singapore (SG)

(21) Appl. No.: 19/118,011

(22) PCT Filed: Oct. 3, 2023
(86) PCT No.: PCT/SG2023/050665

§ 371 Date: Apr. 3, 2025

Related U.S. Application Data
(60) Provisional application No. 63/378,106, filed on Oct. 3, 2022.

(51) Int. Cl. C12N 5/00; B01L 3/00

(52) U.S. Cl. CPC C12N 5/0081; B01L 3/502761; B01L 2200/0652; B01L 2300/0864; B01L 2300/088

(57) ABSTRACT

The invention relates to methods and devices for size-based separation of undifferentiated cells from a population of cells using curvilinear microfluidic channels. The curvilinear microfluidic channels may be a spiral microfluidic channels. The differentiated cells are spinal cord progenitor cells (SCPCs), neural progenitor cells (NPCs) and/or cells differentiated therefrom. The method may comprise flowing an input population of cells through first and second sequentially connected and fluidically communicating curvilinear microfluidic channels, wherein the first channel is coupled to an inlet and the second channel is coupled to one or more outlets, wherein the first curvilinear microfluidic channel has a smaller cross-sectional area than the second curvilinear microfluidic channel.

### TECHNICAL FIELD

The present invention generally relates to methods for cell separation. In particular, the invention relates to methods and devices for size-based separation of undifferentiated cells from a population of cells using curvilinear microfluidic channels.

### BACKGROUND

Pluripotent stem cells such as embryonic stem cells (ESCs) hold great promise for cell therapy as they can be differentiated into many different lineages according to clinical need. The ability to convert a patient's somatic cells into induced pluripotent stem cells (iPSCs) for generating different cell types not only solves the problem of immunological rejection in cell therapy, but also bypasses ethical considerations associated with ESC use. However, the presence of residual iPSCs among differentiated cells poses a clinical risk as these cells can form teratomas and other forms of tumours if transplanted. The potential tumourigenicity of residual iPSCs depends on the number of such cells in the final cell population and also on the nature of the differentiated cell lineages, and will thus differ according to cell manufacturing protocol and clinical application.

Methods of removing residual iPSCs or ESCs have been reported. These include the use of cytotoxic antibodies and small molecule drugs targeting stem cells, fluorescence-activated cell sorting (FACS) using fluorescently-tagged antibodies that recognise stem cell surface markers, and magnetic-activated cell sorting (MACS) using magnetically-conjugated antibodies. In these methods, the small molecules or fluorescent or magnetic markers added may remain as contaminants and subsequently interfere with cell proliferation and behaviour. Moreover, cell viability may be reduced due to the use of high in-line pressures during sorting. These methods also rely heavily on specific surface markers whose expression may vary depending on factors such as the culture medium and differentiation protocol used. Furthermore, the methods can be inefficient and costly to implement at industrial scale for cell manufacturing. There is therefore a need for label-free, efficient and scalable solutions for separating undifferentiated cells from a mixture of cells during cell manufacturing.

It would be desirable to overcome or ameliorate at least one of the above-described problems, or at least to provide a useful alternative.

### SUMMARY

Disclosed herein is a method for removing undifferentiated cells from a population of differentiated and undifferentiated cells, wherein the method comprises: a) providing an input population of differentiated and undifferentiated cells to an inlet of a microfluidic device; b) flowing the input population of cells through at least one curvilinear microfluidic channel coupled to the inlet, wherein the curvilinear microfluidic channel is configured to separate undifferentiated cells from the input population of cells by size; and c) collecting a first output population of cells through one or more outlets coupled to a curvilinear microfluidic channel of the microfluidic device, wherein the first output population of cells has a lower proportion of undifferentiated cells relative to the input population of cells.

Also disclosed herein is a method for purifying differentiated cells from a population of differentiated and undifferentiated cells, wherein the method comprises: a) providing an input population of differentiated and undifferentiated cells to an inlet of a microfluidic device; b) flowing the input population of cells through at least one curvilinear microfluidic channel coupled to the inlet, wherein the curvilinear microfluidic channel is configured to separate differentiated cells from the input population of cells by size; and c) collecting a first output population of cells through one or more outlets coupled to the curvilinear microfluidic channel of the microfluidic device, wherein the first output population of cells has a higher proportion of differentiated cells relative to the input population of cells.

Also disclosed herein is a device for removing undifferentiated cells from a population of differentiated and undifferentiated cells, the device comprising: a) an inlet for receiving an input population of differentiated and undifferentiated cells; b) at least one curvilinear microfluidic channel coupled to the inlet, wherein the curvilinear microfluidic channel is configured to separate undifferentiated cells from the input population of cells by size; and c) one or more outlets coupled to the curvilinear microfluidic channel for collecting an output population of cells, wherein the output population of cells has a lower proportion of undifferentiated cells relative to the input population of cells.

Disclosed herein is a device for purifying differentiated cells from a population of differentiated and undifferentiated cells, the device comprising: a) an inlet for receiving an input population of differentiated and undifferentiated cells; b) at least one curvilinear microfluidic channel coupled to the inlet, wherein the curvilinear microfluidic channel is configured to separate differentiated cells from the input population of cells by size; and c) one or more outlets coupled to the curvilinear microfluidic channel for collecting an output population of cells, wherein the output population of cells has a higher proportion of differentiated cells relative to the input population of cells.

### BRIEF DESCRIPTION OF DRAWINGS

Embodiments of the present invention are hereafter described, by way of non-limiting example only, with reference to the accompanying drawings in which:

FIG. 1: Schematic of a spiral sorting device. It comprises two spiral channels: (1) is connected directly to the inlet and serves to focus the cells on the inner wall (IW); (2) leads directly to the outlets and serves to separate cells based on their size, with larger cells located nearer the inner wall (IW), and smaller cells located nearer the outer wall (OW).

FIG. 2: Average sizes of unsorted and sorted cell populations containing spinal cord progenitor cells (SCPCs) differentiated from induced pluripotent stem cells (iPSCs). (A and B) There are significant differences in average cell size of the “small cell” and “large cell” output populations, and also differences in the average size of cells in these two populations compared to the unsorted population of cells. (C) Cell viability of SCPCs in different size groups before and after cell transplantation. No significant differences observed among the various groups.

FIG. 3: In vivo cell viability of SCPCs injected into rat spinal cord: SCPCs (Unsorted, Sorted-small, Sorted-large, or Mixed) were injected at the T9-T10 level of the rat's spinal cord. Four weeks after implantation, higher cell viability was observed in the Unsorted and the Sorted-small group compared to the Sorted-large or the Mixed group. Sorted-large cells had negligible viable cells. The Mixed group (prepared by mixing the Sorted-small and Sorted-large cells at a ratio of 50:50) showed that the cell viability was reduced by 50% compared to Unsorted or Sorted-small.

FIG. 4: 4 weeks post-injection, none of the injected cells expressed the pluripotent marker, OCT4, and only a small proportion of cells stained positive with Ki-67 and the neural progenitor marker SOX1. No significant differences were observed between the Unsorted and Sorted-small groups.

FIG. 5: 4 weeks post-injection, the transplanted cells exhibited neuronal stem cell markers (HuNu⁺/SOX2⁺) and maintained their spinal cord lineage (hNCAM⁺/HOXB4⁺). No significant differences were observed between the Unsorted and Sorted-small groups.

FIG. 6: (A) Sorted-small cells demonstrated a more extensive or equal extent of neuronal differentiation vs. Unsorted cells after 4 weeks in vivo transplantation into intact rat spinal cord at T9-10 level. More HuNu⁺/NeuN⁺ (indicative of neuronal differentiation) cells were observed in Experiment 1. A similar extent of NeuN expression in Experiment 2. (B) Further confirmation of the observation that Sorted-small cells demonstrated a more extensive or equal degree of neuronal differentiation vs. unsorted cells after 4 weeks in vivo transplantation into intact rat spinal cord at T9-10 level. More hNCAM⁺/NF⁺ (indicative of neuronal differentiation) cells were observed in Experiment 1—a similar extent of NF expression in Experiment 2.

FIG. 7: A sparse population of injected cells differentiated into astrocytes (hNCAM+/GFAP⁺) expressing cells after 4 weeks in vivo. No significant difference was observed between unsorted and sorted-small groups.

### DETAILED DESCRIPTION

This disclosure teaches a method for removing undifferentiated cells from an input population of cells using curvilinear microfluidic channels. The input population of cells may comprise both differentiated and undifferentiated cells. The method may comprise: a) providing an input population of cells to an inlet of a microfluidic device; b) flowing the input population of cells through at least one curvilinear microfluidic channel coupled to the inlet, wherein the curvilinear microfluidic channel is configured to separate undifferentiated cells from the input population of cells by size; and c) collecting a first output population of cells through one or more outlets coupled to the curvilinear microfluidic channel of the microfluidic device, wherein the first output population of cells has a lower proportion of undifferentiated cells relative to the input population of cells.

The disclosure also teaches a method for purifying differentiated cells from a population of differentiated and undifferentiated cells, wherein the method comprises: a) providing an input population of differentiated and undifferentiated cells to an inlet of a microfluidic device; b) flowing the input population of cells through at least one curvilinear microfluidic channel coupled to the inlet, wherein the curvilinear microfluidic channel is configured to separate differentiated cells from the input population of cells by size; and c) collecting a first output population of cells through one or more outlets coupled to the curvilinear microfluidic channel of the microfluidic device, wherein the first output population of cells has a higher proportion of differentiated cells relative to the input population of cells.

Without being bound by theory, the inventors have developed a microfluidic size-based method for separating undifferentiated cells from a mixture of cells. The method is based on the surprising discovery that undifferentiated cells are sufficiently different in size from differentiated cells to be separated using inertial focusing within curvilinear microfluidic channels. For instance, the inventors have found that undifferentiated pluripotent cells can be larger than progenitor cells differentiated therefrom (e.g., neural progenitor cells). The size difference can vary depending on the type and state of differentiation of the differentiated and undifferentiated cells. In general, however, a size difference exists which can be exploited for passive cell separation using inertial microfluidics.

Unlike conventional methods of cell separation like FACS and MACS, the microfluidic method of this disclosure allows for label-free, non-contact, and high throughput removal of undifferentiated cells from a population of differentiated and undifferentiated cells while minimising impact on cell viability and cell function. Microfluidic-based cell separation has the further advantage of being adaptable for resolving a large range of size differences between various types of differentiated and undifferentiated cells through the careful selection of channel geometries and fluidic parameters. The microfluidic method of this disclosure can be automated and integrated into cell manufacturing processes to remove residual undifferentiated cells to reduce the risk of tumour formation following cell implantation.

The method involves the use of microfluidic devices with at least one curvilinear microfluidic channel that is configured to separate undifferentiated or differentiated cells from a mixed population of cells by size. Separation is based on inertial focusing as a result of the curved channel geometry which serves to position and concentrate cells of different sizes at equilibrium positions along the channel.

The term “microfluidic” as used herein, refers to a system or device having one or more fluidic channels, conduits or chambers that are generally fabricated at the millimeter to micrometer scale, e.g., typically having at least one cross-sectional dimension in the range of from about 1 μm to about 1 mm.

The term “channel” as used herein refers to a structure in which fluid may flow. A channel may be a capillary, a conduit, a strip of hydrophilic pattern on an otherwise hydrophobic surface wherein aqueous fluids are confined, etc.

As used herein, a “curvilinear microchannel” is a microchannel in which a longitudinal axis along a direction of flow of the microchannel deviates from a straight line, and may, for example, be a spiral or sinusoidal channel.

As will be appreciated by those of ordinary skill in the art, the channel can be arranged in a variety of shapes (e.g., spiral, multi-looped, serpentine, including a mixture of curved and linear sections) and can have various cross-sectional shapes (e.g., rectangular, trapezoidal) provided that the dimensions of the channel are adapted to separate cells based on cell size, along at least one portion of a cross-section of the at least one curvilinear microchannel.

Without being bound by theory, fluid flowing through curved channels experience both inertial effects and centrifugal forces that push the fluid toward the outer wall of the curved channel. This creates a secondary flow pattern within the channel, known as Dean flow, where fluid moves in the radial direction. Cells suspended in the fluid are subjected to these secondary flows and experience lateral migration. As a result, they are pushed away from the channel walls and, depending on the size of the cells and the flow conditions, they eventually reach equilibrium positions along the channel's cross-section and are concentrated at these regions within the channel. This concentration and alignment of cells away from the channel walls facilitate size-based cell separation.

Without being bound by theory, when a fluid flows through a curved microchannel, particularly one with a spiral shape or profile, the laminar Poiseuille flow is subjected to centrifugal forces. The centrifugal forces disturb the parabolic fluid velocity profile of the laminar flow and the position of maximum fluid velocity shifts from the cross-section centre of the microchannel towards the outer side of the microchannel, causing a sharp velocity gradient to develop between the maximum fluid velocity position and the outer side. The sharp velocity gradient increases fluid pressure and the localised fluid velocity near the outer side is not sufficient to balance this pressure differential. This imbalance is known as Dean instability and leads to recirculation of fluid in the form of two counter rotating Dean vortices in the upper and lower halves of the microchannel. Each Dean vortex loops between the cross-section centre and outer side of the microchannel in order to balance the pressure differential. The Dean vortices or secondary flows are defined by a dimensionless parameter, known as Dean number De, representing the Dean forces due to the secondary flows in the microchannel. The Dean number is defined as De=Re(Dₕ/2r)^{1/2}, where Re is the flow Reynolds number, Dₕ is the hydraulic diameter of the microchannel (which is the smallest dimension of the microchannel and is usually the cross-sectional height of the microchannel), r represents the average radius of curvature of the channel, and δ=Dₕ/2r is the curvature ratio. The hydraulic diameter Dₕ is the cross-sectional diameter for a circular channel or Dₕ=2wh/(w+h) for a rectangular channel (where w and h correspond to width and height of the cross-section respectively).

Cells flowing in a curved microchannel experience Dean drag forces due to the presence of lateral Dean vortices or secondary flows entraining the cells along the flow direction of the Dean vortices. Thus, the cells move repeatedly between the inner and outer sides of the microchannel as the fluid flows along the microchannel. The velocity with which the cells migrate laterally when flowing in the microchannel is dependent on the Dean number De. The lateral distance traversed by a cell can be defined in terms of “Dean cycle”. For example, a cell which is initially positioned near the inner wall of a curved microchannel and migrates to the outer wall of the microchannel at a distance downstream is considered to have completed half a Dean cycle. The cell completes one Dean cycle if it returns back to the original position near the microchannel inner wall further downstream. Thus, depending on the length of the microchannel, the cells can undergo one or more Dean cycles or a fraction of a Dean cycle.

In addition to the Dean drag forces, cells may also experience inertial lift forces, which may be shear-induced due to the parabolic fluid velocity profile and/or wall-induced due to interactions between the cell and the sides or walls of the microchannel. The cells flowing in the microchannel are thus subjected to a combination of inertial lift forces and Dean drag forces. As cell size increases, both the lift force and Dean drag force increase, but with a different power. In the case of the inertial lift force (F_{L}), F_{L}∝a⁴ near the channel centre and F_{L}∝a⁶ near the channel wall where wall effects dominate. In case of the Dean drag force (F_{D}), F_{D}∝a. Thus, larger cells are more significantly influenced by the inertial lift forces while smaller cells are more significantly influenced by the Dean drag forces. As the ratio of the inertial lift forces and Dean drag forces varies for varying particle sizes, the cells can be equilibrated at distinct positions along the microchannel cross-section based on their sizes. Particularly, under the influence of the Dean drag forces which are dominant over inertial lifting forces for smaller cells, the smaller fluid particles initiate migration along the Dean vortices and equilibrate closer to the outer wall of the curved microchannel. For larger cells, inertial lift forces dominate over the Dean drag forces, preventing them from migrating laterally and causing them to equilibrate closer to the inner wall of the curved microchannel. In this manner, cells of different sizes eventually reach equilibrium positions along the channel's cross-section and are concentrated at these regions within the channel. This concentration and alignment of cells facilitate size-based cell separation.

The confinement ratio, defined as CR=a/Dₕ, where a is the particle diameter, is a key parameter to consider for size-based cell separation in a curved microchannel. Generally, for cells to focus and equilibrate at distinct positions along the microchannel cross-section, CR should be at least 0.07 (CR≥0.07).

The flow of the population of cells through the curvilinear microfluidic channel will generally lead to focusing of larger cells towards the inner wall of the channel (i.e., the wall on the radially inner side) and smaller cells towards the opposing outer wall of the channel (i.e., the wall on the radially outer side). Cells of different sizes may then be collected through channel outlets appropriately positioned along the channel cross-section.

### Input Populations of Differentiated and Undifferentiated Cells

In one embodiment, the input population of cells comprises a mixture of cells in suspension. As used herein, “cells in suspension” means cells in a state where they do not adhere tightly to the walls of the vessel (e.g., chamber) in which they are being grown or maintained. In cases where a cell type is grown as an adherent cell it can be treated to provide cells in suspension by the use of various agents such as trypsin. When in suspension, the majority of cells present can be introduced into a microfluidic device for separation by size. In some embodiments, the majority, or greater than about 50%, 60%, 70%, 75%, 80%, 85%, 90% or 95% of the cells in suspension are present as single cells, with higher percentages being preferred.

Differentiated cells and undifferentiated cells in the input population may be derived from human or any of non-human animals. In some embodiments, the differentiated and undifferentiated cells are mammalian cells. In one embodiment, the differentiated and undifferentiated cells are human cells. The human cells may be a cell line or primary cells derived from a patient. In one embodiment, the differentiated cells comprise cells differentiated from the undifferentiated cells in the input population of cells.

As used herein, the term “undifferentiated cells” describes cells characterised by a lack of specific morphological and functional characteristics acquired by normal cells during development (a process known in the art as “differentiation”). Differentiation of cells during development results in different tissues and cell types characterised by different sizes, shapes, metabolic activities, lineage- or cell type-specific markers and/or responsiveness to signals. An undifferentiated cell may be a totipotent, pluripotent, multipotent or monopotent cell. An undifferentiated cell may be a part of a lineage which has never undergone differentiation (e.g., an embryonic stem cell), or derived from a differentiated cell by a process which eliminates the specific morphological and functional characteristics of the differentiated cell (e.g., an induced pluripotent stem cell).

In some embodiments, the undifferentiated cells are stem cells. As used herein, the term “stem cell” refers to a cell which has the ability to self-renew (i.e., to generate an identical copy of itself) and to differentiate into one or more different cell lineages or cell types. Stem cells may be isolated from early developing embryos, from embryonic or adult tissues, or derived from a differentiated cell. Stem cells herein may be totipotent stem cells (which can differentiate into cells from all three germ layers as well as into extraembryonic tissues like the placenta); pluripotent stem cells (which can differentiate into cells from all three germ layers); multipotent stem cells (which can differentiate into a subset of cell types within a particular organ or tissue); oligopotent stem cells (which can differentiate into a few related cell types); or monopotent stem cells (which can differentiate into only one specific cell type).

In some embodiments, the undifferentiated cells are pluripotent cells. As used herein, the term “pluripotent cells” and “pluripotent stem cells” are used interchangeably to describe stem cells which have the potential to differentiate into any of the three germ layers: endoderm, mesoderm or ectoderm. Examples include but are not limited to embryonic stem cells (ESCs) and induced pluripotent stem cells (iPSCs). Pluripotent stem cells may be identified by cell-specific protein markers, e.g., OCT4, SOX2 and NANOG.

In some embodiments, the undifferentiated cells are induced pluripotent stem cells (iPSCs) or human embryonic stem cells (hESCs).

Human embryonic stem (hES) cells can be prepared from human blastocyst cells using techniques well known in the art. Induced pluripotent stem cells (iPSCs) may be generated from any vertebrate species, including humans as well as non-human primates, domestic animals, livestock and other non-human mammals. IPSCs may be generated from any cell type, for example, fibroblast cells, tumour cells, bone marrow cells, stomach cells, liver cells, epithelial cells, follicular cells, connective tissue cells, muscle cells, bone cells, cartilage cells, gastrointestinal cells, splenic cells, kidney cells, lung cells, testicular cells, nervous tissue cells, and blood cells. Such cells can be dedifferentiated by exposure to combinations of transcription factors such as, for example, OCT4, SOX2, KL4, and optionally cMyc, NANOG and/or LIN28. The exposure to transcription factors may be accomplished, e.g., by viral or non-viral methods of application or transduction.

Induced pluripotent stem cells (iPSCs) and human embryonic stem cells (hESCs) may be identified by suitable methods known in the art. For example, the cells may be characterised by their capacity to support teratoma formation in an animal model as a confirmation of pluripotency. Alternatively or additionally, the cells may be identified by genetic and/or DNA methylation profiling, or by the expression of cell type-specific markers.

In some embodiments, the undifferentiated cells are neural stem cells. As used herein, “neural stem cells (NSCs)” describes cells that can generate cell types of the nervous system. NSCs can be characterised by the presence of the markers Nestin, SOX2 and CD133.

In some embodiments, the undifferentiated cells are mesenchymal stem cells. As used herein, “mesenchymal stem cells (MSCs)” are multipotent stem cells that can be isolated from a variety of tissues and can differentiate into a variety of cell types, including osteoblasts (bone cells), chondrocytes (cartilage cells) and adipocytes (fat cells). MSCs have been characterised by a number of surface markers including expression of markers from the following list: CD29, CD44, CD73, CD105, CD106, CD166 and STRO-1.

In some embodiments, the undifferentiated cells are hematopoietic stem cells. As used herein, “hematopoietic stem cells (HSCs)” are multipotent stem cells that give rise to all the blood cell types from the myeloid (monocytes and macrophages, neutrophils, basophils, eosinophils, erythrocytes, megakaryocytes/platelets, dendritic cells), and lymphoid lineages (T-cells, B-cells, NK-cells). Human HSCs have been described as CD34+ CD133+ Lin− cells.

The undifferentiated cells (e.g., ESCs or iPSCs) as described herein may be differentiated into any cell type of interest, including endodermal (giving rise to cells of the endoderm, which gives rise to inner tissues and organs such as the alimentary canal, gut, digestive glands, respiratory system, and intestines/bladder), ectodermal (giving rise to cells of the ectoderm, which gives rise to the nervous system, skin, and other outermost specialized tissues and organs), mesodermal (giving rise to cells of the mesoderm; mesoderm is the middle germ layer of an embryo coming from the inner cell mass of the blastocyst; it gives rise to bone, muscle, connective tissues, including the dermis, the blood vascular system, the urogenital system except the bladder, and contributes to some glands), neuroectodermal (giving rise to any cells of the neuroectoderm, which gives rise to neurons, supporting cells, and ependyma of the central nervous system and the neural crest cells that form peripheral ganglia and a wide variety of other tissues), neural (giving rise to any cells of the nervous system peripheral and central; autonomic and somatic, including all neurons, support cells/glia, etc).

In some embodiments, the undifferentiated cells are differentiated into a population of cells, for example, a cobblestone-like cell line, a cardiomyocytic cell population, an epithelial cell population such as a keratin-containing or gut-like epithelial cell population, a gastrointestinal cell population, a respiratory cell population, a hepatic cell population, a pancreatic cell population, an endocrinic cell population, an epidermal cell population, a myogenic cell population, a cartilage cell population, a mucosal cell population, a skeletal cell population, a cartilage cell population, a nephritic cell population, a lymphatic cell population, a splenic cell population, a neural cell population, a hematopoietic cell population, or the precursors of any of the preceding.

As used herein, “differentiated cells” are cells which are at a later stage of differentiation than the undifferentiated cells in the input population of cells. In preferred embodiments, the differentiated cells lack pluripotency, for instance, the cells do not express one or more protein markers specific to pluripotent cells, such as OCT4, SOX2 and/or NANOG. The differentiated cells may be a cell from any one of endodermal, mesodermal or ectodermal lineage. For the avoidance of doubt, a differentiated cell of this disclosure may be capable of further differentiation into one or more lineages, or one or more cell types.

Cells of endodermal, mesodermal and ectodermal lineage are widely known in the art and easily identifiable by a skilled person, for example, by the presence of cell-specific morphological or functional characteristics, or by the expression of lineage-specific markers. By way of non-limiting example, endodermal cell markers may include SOX17, GATA4, FOXA2, CK8, CK18, CDX2, TTF-1 and HNF4α, among others; mesodermal cell markers can include Brachyury, Nkx2.5, desmin, CD34, vimentin, MyoD, etc.; and ectodermal cells markers can include PAX6, Nestin, NCAM, KRT10, KRT14, GFAP, etc.

In one embodiment, the differentiated cells are cells of endodermal lineage. Cells of endodermal lineage include but are not limited to epithelial cells of the gastrointestinal tract, cells of the respiratory epithelium (e.g., alveolar cells and cells lining the bronchi) cells of the thyroid and parathyroid, cells of the thymus and tonsils, liver cells (e.g., hepatocytes), pancreatic cells (e.g., beta cells), cells lining the bladder and urethra, and progenitor cells of the above.

In one embodiment, the differentiated cells are cells of mesodermal lineage. Cells of mesodermal lineage include but are not limited to hematopoietic stem cells, mesenchymal stem cells, blood cells (e.g., myeloid and lymphoid cells), vascular endothelial cells, dermal cells, muscle cells (e.g., skeletal, smooth and cardiac muscle cells), bone cells (e.g., osteoblasts), cartilage cells (e.g., chondrocytes), cells of connective tissue (e.g., fibroblasts, adipocytes, etc.), cells of the male and female reproductive systems, kidney cells (e.g., renal epithelial cells), cells of the spleen, and progenitor cells of the above.

In one embodiment, the differentiated cells are cells of ectodermal lineage. Cells of ectodermal lineage include but are not limited to neural stem cells, cells of the nervous system (e.g., neurons, glial cells), epidermal cells (e.g., keratinocytes), cells of the mouth (e.g., buccal cells lining the mouth, nostrils and anus), melanocytes, glandular cells of the mammary glands, sweat glands and pineal gland, adrenal medullary cells, cells of the visual, auditory and olfactory sense organs, and progenitor cells of the above.

In some embodiments, the differentiated cells are multipotent cells. In some embodiments, the differentiated cells are oligopotent cells. In some embodiments, the differentiated cells are monopotent cells. In some embodiments, the differentiated cells are terminally differentiated cells. In some embodiments, the differentiated cells are capable of undergoing passage in culture without observed replicative crisis, up to and including days, weeks, months and years of passage in cell culture. In some embodiments, the differentiated cells are incapable of undergoing passage in culture without observed replicative crisis. In each case, the ordinary skilled person can readily assess the viability and lineage potency of the derived cell population using methods known in the art.

Generally, specific stem or progenitor cells may be identified according to whether they express one or more markers associated with such cells. For example, markers of human ESCs include but are not limited to OCT4, NANOG, TRA-1-81, SSEA-4, and SSEA-3. Markers of NSCs and neural progenitor cells (NPCs) include without limitation Nestin, GFAP, Musashi-1, Sox1, Sox2, Pax6 and CD133. In addition, an ESC that is maintained in an undifferentiated state generally will not express, or will express relatively low levels of markers indicative of differentiation such as Brachyury, Sox17, FoxA2, Pax6, Otx2, and Sox1. Markers such as SSEA-4, OCT4, Brachyury and Sox17, as well as other markers indicative of differentiated and undifferentiated states, are known and routinely used in the art.

In one embodiment, the differentiated cells are spinal cord progenitor cells (SCPCs), neural progenitor cells (NPCs) and/or cells differentiated therefrom. NPCs are found throughout the nervous system, including the brain and spinal cord, and have the potential to differentiate into a wide range of neural cell types, including neurons and glial cells. NPCs may be identified by cellular markers including, NF200, Sox1, NCAM, GFAP. SCPCs are a subset of NPCs located within the spinal cord and have a more restricted differentiation potential than NPCs, primarily giving rise to cell types found in the spinal cord, e.g., motor neurons, interneurons and glial cells that support spinal cord function. SCPCs may additionally be identified by the markers Olig2 and Nkx6.1, Nkx6.2 and HOX genes (e.g., HoxB4). Cells differentiated from SCPCs or NPCs include, for example, neurons and glial cells (e.g., astrocytes, oligodendrocytes, ependymal cells, microglia, Schwann cells, satellite cells, etc.).

In one embodiment, the differentiated cells are T cells, NK cells or NKT cells. These cells may be differentiated from pluripotent cells or from hematopoietic stem cells. T cells express the T cell receptor (TCR) on their cell surface as well as CD3 and CD4 or CD8. Markers including CD56 and CD16 may be used to identify and distinguish NK cells from other lymphocytes. NKT cells express both TCR and NK cell markers.

The differentiated and undifferentiated cells may vary in size depending on the type of cell, the stage of differentiation of the cell, and the source of the cell. In some embodiments, the sizes for the differentiated and undifferentiated cells in the input population are determined prior to microfluidic separation. The sizes may be used to adjust channel geometries and/or operating fluidic parameters to increase the efficiency and/or resolution of cell sorting. Size ranges for various types of differentiated and undifferentiated cells are known in the art. Table 1 provides size ranges for some of these cells. Multipotent stem cells (e.g., neural and mesenchymal stem cells) may either be undifferentiated cells (e.g., if used to derive differentiated progeny) or differentiated cells (e.g., if differentiated from pluripotent cells) for the purposes of the methods herein. The skilled person can also determine the sizes of differentiated and undifferentiated cells, for example, by measuring the cell sizes of pure or substantially pure differentiated or undifferentiated cell populations. Such cell populations may be isolated and identified by methods known in the art, e.g., through immunolabelling of cell-specific markers.

In another embodiment, the average cell size for the input population of cells is determined prior to microfluidic separation. This size is used to adjust channel and microfluidic parameters for a first round of size-based separation. In this first round of separation, the mixed population of differentiated and undifferentiated cells is sorted into subpopulations of cells with defined size ranges. These subpopulations may contain different proportions of differentiated and undifferentiated cells. Cell types in different subpopulations are then determined (e.g., by morphology, functional assays and/or immuno- or other types of cellular staining) and the sizes of the different cell types are measured. The subpopulations are then subjected to second or subsequent rounds of size-based sorting with operating parameters adjusted based on the measured cell sizes. This embodiment allows for iterative cell separation without having to pre-determine the sizes of the differentiated and undifferentiated cells.

By way of non-limiting example, the undifferentiated cells may have a diameter of between about 7 μm to about 30 μm, The undifferentiated cells may have a diameter of about 7 μm, about 7.5 μm, about 8 μm, about 8.5 μm, about 9 μm, about 9.5 μm, about 10 μm, about 10.5 μm, about 11 μm, about 11.5 μm, about 12 μm, about 12.5 μm, about 13 μm, about 13.5 μm, about 14 μm, about 14.5 μm, about 15 μm, about 15.5 μm, about 16 μm, about 16.5 μm, about 17 μm, about 17.5 μm, about 18 μm, about 18.5 μm, about 19 μm, about 19.5 μm, about 20 μm, about 20.5 μm, about 21 μm, about 21.5 μm, about 22 μm, about 22.5 μm, about 23 μm, about 23.5 μm, about 24 μm, about 24.5 μm, about 25 μm, about 25.5 μm, about 26 μm, about 26.5 μm, about 27 μm, about 27.5 μm, about 28 μm, about 28.5 μm, about 29 μm, about 29.5 μm, or about 30 μm.

By way of non-limiting example, the differentiated cells may have a diameter of between about 7 μm to about 30 μm, The undifferentiated cells may have a diameter of about 7 μm, about 7.5 μm, about 8 μm, about 8.5 μm, about 9 μm, about 9.5 μm, about 10 μm, about 10.5 μm, about 11 μm, about 11.5 μm, about 12 μm, about 12.5 μm, about 13 μm, about 13.5 μm, about 14 μm, about 14.5 μm, about 15 μm, about 15.5 μm, about 16 μm, about 16.5 μm, about 17 μm, about 17.5 μm, about 18 μm, about 18.5 μm, about 19 μm, about 19.5 μm, about 20 μm, about 20.5 μm, about 21 μm, about 21.5 μm, about 22 μm, about 22.5 μm, about 23 μm, about 23.5 μm, about 24 μm, about 24.5 μm, about 25 μm, about 25.5 μm, about 26 μm, about 26.5 μm, about 27 μm, about 27.5 μm, about 28 μm, about 28.5 μm, about 29 μm, about 29.5 μm, or about 30 μm.

TABLE 1
Size ranges for differentiated and undifferentiated cells.

| Cell type | Diameter (μm) |
|---|---|
| Induced pluripotent stem cell | 10-20 |
| Embryonic stem cell | 10-20 |
| Neural stem cell | 10-20 |
| Mesenchymal stem cell | 15-30 |
| Hematopoietic stem cell | 8-12 |
| Neural progenitor cell | 10-20 |
| Spinal-cord progenitor cell | 10-20 |

In some embodiments, the size difference between the undifferentiated cells and the differentiated cells is at least about 0.4 μm. The size difference between the undifferentiated cells and the differentiated cells can be, for example, at least about 0.4 μm, at least about 0.6 μm, at least about 0.8 μm, at least about lum, at least about 1.2 μm, at least about 1.4 μm, at least about 1.6 μm, at least about 1.8 μm, at least about 2 μm, at least about 2.2 μm, at least about 2.4 μm, at least about 2.6 μm, at least about 2.8 μm, at least about 3 μm, at least about 3.2 μm, at least about 3.4 μm, at least about 3.6 μm, at least about 3.8 μm, at least about 4 μm, at least about 4.2 μm, at least about 4.4 μm, at least about 4.6 μm, at least about 4.8 μm, at least about 5 μm, at least about 5.2 μm, at least about 5.4 μm, at least about 5.6 μm, at least about 5.8 μm, at least about 6 μm, at least about 6.2 μm, at least about 6.4 μm, at least about 6.6 μm, at least about 6.8 μm, at least about 7 μm, at least about 7.2 μm, at least about 7.4 μm, at least about 7.6 μm, at least about 7.8 μm, at least about 8 μm, at least about 8.2 μm, at least about 8.4 μm, at least about 8.6 μm, at least about 8.8 μm, at least about 9 μm, at least about 9.2 μm, at least about 9.4 μm, at least about 9.6 μm, at least about 9.8 μm, or at least about 10 μm.

In some embodiments, the size difference between the undifferentiated cells and the differentiated cells is about 0.4 μm, about 0.6 μm, about 0.8 μm, about 1 μm, about 1.2 μm, about 1.4 μm, about 1.6 μm, about 1.8 μm, about 2 μm, about 2.2 μm, about 2.4 μm, about 2.6 μm, about 2.8 μm, about 3 μm, about 3.2 μm, about 3.4 μm, about 3.6 μm, about 3.8 μm, about 4 μm, about 4.2 μm, about 4.4 μm, about 4.6 μm, about 4.8 μm, about 5 μm, about 5.2 μm, about 5.4 μm, about 5.6 μm, about 5.8 μm, about 6 μm, about 6.2 μm, about 6.4 μm, about 6.6 μm, about 6.8 μm, about 7 μm, about 7.2 μm, about 7.4 μm, about 7.6 μm, about 7.8 μm, about 8 μm, about 8.2 μm, about 8.4 μm, about 8.6 μm, about 8.8 μm, about 9 μm, about 9.2 μm, about 9.4 μm, about 9.6 μm, about 9.8 μm, about 10 μm. In one embodiment, the size difference between undifferentiated pluripotent cells and differentiated SCPCs is about 4 μm.

The input population of cells may be introduced or perfused into the inlets using known devices such as syringes and/or fluid pumps, for example, piston pumps, gear pumps, peristaltic pumps, piezoelectric micropumps, or using a controllable pressure regulator.

In some embodiments, the method removes at least about 10%, at least about 20%, at least about 30%, at least about 40%, at least about 50%, at least about 60%, at least about 70%, at least about 80%, or at least about 90% of undifferentiated cells from the input population of cells. In some embodiments, the method removes about 10%, about 20%, about 30%, about 40%, about 50%, about 60%, about 70%, about 80%, about 90%, or about 100% of undifferentiated cells from the input population of cells.

In some embodiments, the method purifies at least about 10%, at least about 20%, at least about 30%, at least about 40%, at least about 50%, at least about 60%, at least about 70%, at least about 80%, or at least about 90% of differentiated cells from the input population of cells. In some embodiments, the method purifies about 10%, about 20%, about 30%, about 40%, about 50%, about 60%, about 70%, about 80%, about 90%, or about 100% of differentiated cells from the input population of cells.

In some embodiments, the viability of the differentiated cells in the input population of cells and in the first output population of cells is comparable. For example, the method may lead to at most a 10% decrease, at most a 9% decrease, at most an 8% decrease, at most a 7% decrease, at most a 6% decrease, at most a 5% decrease, at most a 4% decrease, at most a 3% decrease, at most a 2% decrease, or at most a 1% decrease in the viability of the differentiated cells. Cell viability may be measured by methods known in the art, e.g., using metabolic assays, proliferation assays, or colorimetric or fluorescent stains that differentially stain living and dead cells.

In some embodiments, the differentiation potential of the differentiated cells in the input population of cells and in the first output population of cells is comparable. For example, the method may lead to at most a 10% decrease, at most a 9% decrease, at most an 8% decrease, at most a 7% decrease, at most a 6% decrease, at most a 5% decrease, at most a 4% decrease, at most a 3% decrease, at most a 2% decrease, or at most a 1% decrease in the differentiation potential of the differentiated cells. Cell differentiation potential may be measured using differentiation assays, e.g., by determining the proportion of different cell types that are generated after exposing the differentiated cells to specific differentiation conditions or factors.

### Dimensional Properties of Microfluidic Channels

A curvilinear channel for the methods herein may have multiple curvatures. In one embodiment, the curvilinear channel is a spiral. Spiral microchannels can comprise one or more loops. In certain aspects, each spiral microchannel can independently be a 2-loop microchannel, a 3-loop microchannel, a 4-loop microchannel, a 5-loop microchannel, a 6-loop microchannel, a 7-loop microchannel, an 8-loop microchannel, a 9-loop microchannel, a 10-loop microchannel, etc. It will be appreciated that the spiral may be oriented in a clockwise or anti-clockwise manner. The inlet can be provided at the outermost loop of the spiral or at the innermost loop.

In certain examples, a spiral microchannel can have a radius of curvature in a range of between about 2.5 mm and about 25 mm. For example, the spiral microchannel can have a radius of curvature of about 2.5 mm, 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, or about 25 mm. In one embodiment, the spiral microchannel has a radius of curvature that decreases from about 15 mm at the outermost loop to about 5 mm at innermost loop.

The width of a curvilinear channel for use in the disclosed methods can be in a range of between about 100 μm and about 2000 μm, such as about 100 μm, about 200 μm, about 300 μm, about 400 μm, about 500 μm, about 600 μm, about 700 μm, about 800 μm, 900 μm, about 1000 μm, about 1100 μm, about 1200 μm, about 1300 μm, about 1400 μm, about 1500 μm, about 1600 μm, about 1700 μm, about 1800 μm, about 1900 μm, or about 2000 μm. The width of the channel may be uniform throughout the channel length. In one embodiment, the width of a curvilinear channel is about 800 μm.

The height of a curvilinear channel can be in a range of between about 20 μm and about 300 μm, such as about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm.

In some embodiments, the curvilinear channel has a non-uniform cross-sectional height. For example, the channel may have a trapezoidal cross-section defined by a radially inner wall, a radially outer wall, a bottom wall and a top wall, with the radially inner wall and the radially outer wall being unequal in height. The height of the radially inner wall may be smaller or larger than the height of the radially outer wall. Microfluidic channels with a trapezoidal cross-section are preferred because the maximum velocity is asymmetric along the channel cross-section resulting in the formation of stronger Dean vortex cores skewed towards the deeper channel side. This is capable of increasing the separation distance between cells of different sizes even in a one-inlet configuration without sheath flow. In a trapezoidal channel, as shown in WO2014/046621, particles focus near the inner channel wall at low flow rates (similar to channels with rectangular cross-section), while beyond a certain threshold flow rate, they switch to an equilibrium position located at the outer half. Other factors can affect the focusing position and separation efficiency, such as the width of the microchannel, inner and outer height of the microchannel cross-section, the radius of the spiral curvature, and the slant angle.

In one embodiment, a curvilinear channel has a trapezoidal cross-section, wherein the height of the radially inner wall is smaller than the height of the radially outer wall. The height of the radially inner side can be in a range of between about 20 μm and about 200 μm, such as about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm. The height of the radially outer side can be in a range of between about 50 μm and about 300 μm, such as about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm.

The slant angle is the angle between the top of the shorter inner wall and the top of the taller outer wall. The slant angle can be in a range of between about 2 degrees and about 60 degrees. Thus, the slant angle can be about 2 degrees, about 4 degrees, about 6 degrees, about 8 degrees, about 10 degrees, about 12 degrees, about 14 degrees, about 16 degrees, about 18 degrees, about 20 degrees, about 22 degrees, about 24 degrees, about 26 degrees, about 28 degrees, about 30 degrees, about 32 degrees, about 34 degrees, about 36 degrees, about 38 degrees, about 40 degrees, about 42 degrees, about 42 degrees, about 46 degrees, about 48 degrees, about 50 degrees, about 52 degrees, about 54 degrees, about 56 degrees, about 58 degrees, or about 60 degrees. The slant angle of a trapezoidal channel affects the focusing behavior in two ways: (i) the threshold flow rate required to trap cells in the Dean vortex as a function of particle size and (ii) the location of the Dean vortex core. A large slant angle (i.e., in a range of between about 10 degrees and about 60 degrees) will lead to strong Dean flow at the outer side and increase the cell trapping capability. A large slant angle can also decrease the threshold flow rate required to trap cells of a given size within the Dean vortex.

As used herein, an “aspect ratio” is the ratio of a channel's height divided by its width and provides the appropriate cross-section of the channel to isolate cells in the input population, based on cell size, along at least one portion of a cross-section of the at least one curvilinear microchannel.

The aspect ratio of the channel can be in a range of between about 0.1 and about 1, such as about 0.1, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59, about 0.6, about 0.61, about 0.62, about 0.63, about 0.64, about 0.65, about 0.66, about 0.67, about 0.68, about 0.69, about 0.7, about 0.71, about 0.72, about 0.73, about 0.74, about 0.75, about 0.76, about 0.77, about 0.78, about 0.79, about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, about 0.99, or about 1.

The length of a curvilinear channel can be equal to or greater than about 3 cm, such as about 5 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, or about 100 cm.

In some embodiments, the method comprises flowing the input population of cells through first and second sequentially connected and fluidically communicating curvilinear microfluidic channels, wherein the first channel is coupled to the inlet and the second channel is coupled to one or more outlets. The first and second channels may have different geometries giving rise to different confinement ratios (CR) to enable more effective size-based cell separation.

In some embodiments, the first curvilinear microfluidic channel has a smaller cross-sectional area than the second curvilinear microfluidic channel. For example, when both channels have a rectangular cross-section, the width and/or height of the first channel may be less than that of the second channel. In another example, where the first channel has a rectangular cross-section and the second spiral microchannel has a trapezoidal cross-section, the cross-sectional area of the first spiral microchannel may be less than that of the second channel.

Cells entering the first microchannel with smaller cross-sectional area can have a relatively large CR because the dimensions or cross-sectional area of the first spiral microchannel are small. This may permit cells to be concentrated into a band near a channel wall as they pass through the first microchannel. This concentrated stream of cells then enters the second microchannel, where the cell's CR value decreases due to the increased channel size, resulting in the equilibrium locations shifting along the channel cross-section and the cells forming concentrated bands at different equilibrium locations depending on their sizes.

In some embodiments, the first and second curvilinear channels are both spiral channels.

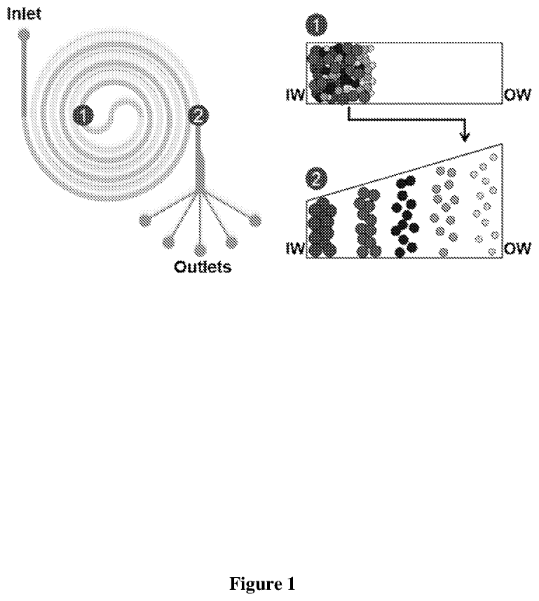

In one embodiment, the first spiral microchannel and the second spiral microchannel are nested together, for example, forming a Fermat spiral. The transition region may be S-shaped. The first spiral microchannel can spiral in a clockwise or counter-clockwise direction and the second spiral microchannel can spiral in the same or in the opposite direction to that of the first spiral microchannel. The first spiral microchannel can, for example, spiral in the counter-clockwise direction, change direction at the transition region (for example, in the S-shaped transition region), and then the second spiral microchannel spirals in the clockwise direction (e.g., see FIG. 1). Alternatively, the first spiral microchannel can spiral in the clockwise direction, change direction at the transition region, and then second spiral microchannel spirals in the counter-clockwise direction.

In one embodiment, the second spiral microchannel runs parallel to the first microchannel. In another embodiment, the second spiral microchannel is positioned over or under the first spiral microchannel.

Depending on the configurations of the spiral microchannels, the inlet can be on the circumference or periphery (outside of the spiral) of the first spiral channel or on the inside or centre of the first channel. In addition, depending on the configuration of the spiral microchannels, the one or more outlets can be on the circumference (outside of the spiral) of the second channel or on the inside of the second channel. In some embodiments with nested first and second spiral microchannels, and the inlet and the outlets are on the circumference of the channel.

The first and second spiral microchannels can each independently have a uniform cross-sectional height or a non-uniform cross-sectional height. For example, the first and second microchannels can both have a rectangular cross-section. In another example, the first and second microchannels can both have a non-rectangular cross-section, for example, both microchannels can have a trapezoidal cross-section. In yet another example, the first microchannel has a rectangular cross-section and the second microchannel has a non-rectangular (e.g., a trapezoidal) cross-section. Microfluidic systems with non-rectangular cross-sections are described, for example, in WO2014/046621, the contents of which are incorporated by reference herein. By designing appropriate channel geometries parameters, small cells may be focused towards the outer wall of the curved or spiral microchannel, and larger cells focused towards the inner wall.

### Fluidic Properties of Microfluidic Channels

As described above, the inertial lift forces F_{L} and Dean drag forces F_{D} vary for cells of different sizes. Particularly, both forces F_{L} and F_{D} scale differently with cell size, and the superposition of F_{L} and F_{D} determines the equilibrium position of the cell within the channel cross-section. Thus, by calibrating the microchannel dimensions (e.g., the hydraulic radius Dₕ and radius of curvature r) and fluid velocity according to the particle size of the target differentiated and/or undifferentiated cells, the skilled person can estimate the F_{L} and F_{D} experienced by the target cells and determine approximate equilibrium positions for cells of a particular size. The use of computational flow models may aid with estimating equilibrium positions. The target cells can be equilibrated at distinct positions along the microchannel cross-section based on the superposition of F_{L} and F_{D}, thereby facilitating separation and isolation of the undifferentiated cells.

In some embodiments, the input population of cells is provided at a cell density of between about 105 to about 10⁷ cells/ml. For example, the cells may be provided at a cell density of about 10⁵ cells/ml, about 10⁶ cells/ml, or about 10⁷ cells/ml. Higher cell densities may lead to cell-cell interactions and cell-channel wall interactions that affect focusing behavior.

The flow rate of fluid through the micro-fluidic device will vary depending on channel geometry, cell size, etc. Exemplary flow rates can be in a range of between about 0.5 ml/min and about 100 ml/min, such as between about 0.5 ml/min and about 20 mL/min, or between about 0.5 mL/min and about 10 mL/min. The flow rate may be about 0.5 ml/min, about 1 ml/min, about 2 ml/min, about 3 ml/min, about 4 ml/min, about 5 ml/min, about 6 ml/min, about 7 ml/min, about 8 ml/min, about 9 ml/min, about 10 ml/min, about 11 ml/min, about 12 ml/min, about 13 ml/min, about 14 ml/min, about 15 ml/min, about 16 ml/min, about 17 ml/min, about 18 ml/min, about 19 ml/min, or about 20 ml/min. Higher flow rates may be used for separating populations of larger cells, and lower flow rates used to resolve populations of smaller cells.

### Collection Outlets

Cells may be collected at one or more outlets at the end of a curvilinear channel. The outlets may be arranged to provide output cell populations with a particular size range. For example, larger cells which may be focused closer to the inner wall of a curved or spiral channel may be collected from outlets closer to the inner wall, and smaller cells which may be focused closer to the outer wall of a channel may be collected from outlets near the outer wall.

In preferred embodiments the device has at least two outlets. One outlet provides a first output population of cells which is relatively depleted of undifferentiated cells compared to the input population. Depending on channel geometry and on the relative size differences between the undifferentiated and differentiated cells, this outlet may be situated nearer the inner or the outer wall. By way of non-limiting example, when the input cell population contains undifferentiated cells which are larger than differentiated cells, and the device contains spiral channels with a trapezoidal cross-section where the inner wall is smaller than the outer wall, the first output population (which is enriched in the smaller differentiated cells) may be collected from an outlet near the outer wall of the channel.

In some embodiments, the method herein comprises collecting a second output population of cells through the one or more outlets of the microfluidic device, wherein the second output population of cells has a higher proportion of undifferentiated cells relative to the input population of cells.

In other embodiments, the method herein comprises repeating steps (a) to (c) at least once, wherein the first output population of cells is provided as the input population of cells in the first and subsequent repeats. This recirculation of cells may further deplete the cell population of undifferentiated cells and/or concentrate the differentiated cells.

In certain embodiments, the device comprises a system for closed-loop recirculation of cells. The closed-loop recirculation system may comprise a syringe in fluid communication with the inlet of the curvilinear channel and a first outlet providing the first output population; a first check valve positioned between and in fluid communication with the first outlet and the syringe; a syringe in fluid communication with the first outlet and the inlet of the curvilinear channel; and a second check valve positioned between and in fluid communication with the syringe and the inlet of the curvilinear channel.

A check valve permits only one direction of flow while the opposite direction of flow is blocked, for example, by an internal membrane. The first check valve permits flow in the direction from the first outlet to the syringe and blocks flow in the direction from the syringe to the first outlet. The first check valve can comprise an inner membrane that blocks flow in the direction from the syringe to the first outlet when the syringe is actuated to infuse the fluid into the inlet of channel. The second check valve permits flow in the direction from the syringe to the inlet of the microchannel and blocks flow in the direction from the inlet of the microchannel to the syringe. The second check valve can comprise an inner membrane that blocks flow in the direction from the inlet of the microchannel to the syringe when the syringe is actuated to withdraw the fluid from the first outlet into the syringe.

Thus, for a device with closed-loop recirculation, the method may comprise: providing an input population of differentiated and undifferentiated cells to an inlet of a microfluidic device; flowing the input population of cells through at least one curvilinear microfluidic channel coupled to the inlet, wherein the curvilinear microfluidic channel is configured to separate undifferentiated cells from the input population of cells by size; collecting a first output population of cells through a first outlet coupled to a curvilinear microfluidic channel of the microfluidic device, wherein the first output population of cells has a lower proportion of undifferentiated cells relative to the input population of cells; recirculating the first output population of cells by actuating the syringe to withdraw the first output population from the first outlet and infusing the first output population into the inlet of the curvilinear microchannel.

As referred to herein, actuation of the syringe can refer to withdrawal motion (e.g., withdrawing fluid from one of the outlets) and/or infusion motion (e.g., infusion into the inlet of a curvilinear channel). Back-and-forth motions (in other words, withdrawal and infusion motions) of the syringe and/or syringe pumps result in recirculation of cell populations from the first outlet into the inlet of the microchannel. Each time all or substantially all of the cells in the first outlet is recirculated in the device, a cycle of recirculation is completed. The population of cells collected after being directed through the device (either after first passage through the device or after one or more cycles of recirculation) can be referred to herein as the “final output population”. The methods described herein can comprise no cycle of recirculation or one or more cycles of recirculation. In certain aspects, the method entails one, two, three, four, five, six, seven, eight, nine or ten cycles of recirculation. The number of cycles of recirculation can depend on a number of factors including, but not limited to, the desired cell separation in the final output population, the desired cell purity in the final output, the desired cell concentration in the final output, time of operation, the number of devices, etc.

### Microfluidic Device

Disclosed herein is a device for removing undifferentiated cells from a population of differentiated and undifferentiated cells, the device comprising: a) an inlet for receiving an input population of differentiated and undifferentiated cells; b) at least one curvilinear microfluidic channel coupled to the inlet, wherein the curvilinear microfluidic channel is configured to separate undifferentiated cells from the input population of cells by size; and c) one or more outlets coupled to the curvilinear microfluidic channel for collecting an output population of cells, wherein the output population of cells has a lower proportion of undifferentiated cells relative to the input population of cells.

Also disclosed herein is a device for purifying differentiated cells from a population of differentiated and undifferentiated cells, the device comprising: a) an inlet for receiving an input population of differentiated and undifferentiated cells; b) at least one curvilinear microfluidic channel coupled to the inlet, wherein the curvilinear microfluidic channel is configured to separate differentiated cells from the input population of cells by size; and c) one or more outlets coupled to the curvilinear microfluidic channel for collecting an output population of cells, wherein the output population of cells has a higher proportion of differentiated cells relative to the input population of cells.

In accordance with an embodiment of the invention, microchannels and devices, including spiral microchannels, which may be used that are taught in PCT Application No. PCT/US2019/061479, the entire disclosure of which is incorporated herein by reference.

The microfluidic device may be fabricated or manufactured in a polycarbonate (PC) material by injection molding using suitable master molds, as will be readily understood by the skilled person. Alternatively, the device may be fabricated using poly-dimethylsiloxane (PDMS) using standard micro-fabrication soft-lithographic techniques known in the art. Other materials that may be used to fabricate or manufacture the microfluidic device include, but are not limited to, glass, polystyrene (PS), poly(methyl methacrylate) (PMMA), and Cyclic Olefin Copolymer (COC).

As will be appreciated by those skilled in the art, the microfluidic device can further comprise other components upstream, downstream, or within a device. For example, one or more microfluidic devices can further comprise one or more collection devices (e.g., a reservoir), flow devices (e.g., a syringe, pump, pressure gauge, temperature gauge), analysis devices (e.g., a 96-well microtitre plate, a microscope), filtration devices (e.g., a membrane), e.g., for upstream or downstream analysis (e.g., immunostaining, polymerase chain reaction (PCR) such as reverse transcription PCR, quantitative PCR, fluorescence such fluorescence in situ hybridization (FISH)), sequencing, and the like. An imaging system may be connected to the device, to capture images from the device, and/or may receive light from the device, in order to permit real time visualisation of the separation process and/or to permit real time enumeration of isolated cells. In one example, the imaging system may view and/or digitise the image obtained through a microscope when the device is mounted on a microscope slide. For instance, the imaging system may include a digitiser and/or camera coupled to the microscope and to a viewing monitor and computer processor. In certain aspects, the device comprises a pump such as a syringe pump, a pressure pump, a peristaltic pump, or a combination of any of thereof. In certain aspects, the device is portable.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Example 1: Fabrication of Multidimensional Double Spiral (MDDS) Sorting Device

Plastic spiral devices were fabricated by the standard injection molding method. The plastic spiral devices were designed with specific channel dimensions by the 3D CAD software (SolidWorks 2020). For injection molding of the plastic spiral devices, top (flat surface w/or w/o the protruded connection ports) and bottom (channel side) master molds were fabricated via a micro-milling process (RnD Factory, South Korea) and duralumin was used for the mold material. The top master mold having the protruded connection ports was used to fabricate the plastic device for tubing connection as the top layer of the stacked device (or when a not-stacked single device is used alone), and the top master mold without the connection ports was used to fabricate the plastic device for the device stacking while the same bottom master mold was used for both connection types to imprint the same spiral channel. Polycarbonate resin was used for the plastic device material. For sealing the channel side of the plastic spiral device, double-sided adhesive film (IS-00726-01, RnD Factory, South Korea) was used. Processes of cutting the film to have the same size as the plastic device and making holes on the film for fluidic access were conducted precisely and automatically by a film cutting machine (GSP32-32, Gu Sung Machine, South Korea). The manufactured plastic spiral device was bonded to the double-sided adhesive film via a manual film attachment process using an alignment-guide plate in clean-room space. To fabricate the multiplexed spiral device, a plastic device having protruded connection ports was used for the top layer of the stacked device for tubing connection, and plastic devices without connection ports were used for the device stacking. For precise and fast alignment for stacking, an aluminum pin holder was fabricated via a standard milling process. To prevent fluidic leakage, the multiplexed spiral device was clamped by the top and bottom stainless-steel plates (5 mm thickness) with bolted joints.

### Example 2: Size-Based Separation of Spinal Cord Progenitor Cells (SCPCs) from Induced Pluripotent Stem Cells (iPSCs)

### Materials and Methods

Spinal Cord Progenitor Cell (SCPC) Differentiation from Human iPSCs

Human induced pluripotent stem cells (iPSCs) were maintained in StemMACS iPS-Brew XF cell culture medium on Matrigel Matrix coating (Corning, USA). The cord lining epithelial cells (CLEC) derived iPSCs were developed by CellResearch Corporation and are hypothesized to have the immune-privileged properties of the CLECs. The iPSCs were routinely passaged every 5-7 days using ReLeSR™ Passaging Reagent (Stemcell Technologies, Canada). The iPSCs were then differentiated into SCPCs based on a modified protocol to generate spinal motor neurons. Briefly, when the iPSCs were 70-80% confluent, the iPSCs were dissociated into single-cell suspension using Accutase (Nacalai Tesque Inc., Japan). The dissociated iPSCs were then counted and reseeded at a density of 800,000 cells per well in a 6-well plate in a neural induction medium supplemented with ROCK inhibitor Y-27632 (5 μM). The neural induction medium consisted of DMEM/F12 (50%, Biological Industries, Israel), neural medium (50%), NeuroBrew-21 (1×), N2 (1×), Non-Essential Amino Acid (1×, Thermo Fisher Scientific, USA), Glutamax (0.5×, Thermo Fisher Scientific), LDN-193189 (0.5 μM), and CHIR-99021 (4.25 μM). On day 3, retinoic acid (RA, 10 μM, Sigma-Aldrich, USA) was added to the medium. On day 4, the cells were reseeded at a lower density (2 million cells per 100 mm dish) in the same medium and maintained with daily medium change until day 10. On day 10, the cells were characterized and termed as SCPCs, which were then frozen or used for subsequent experiments. Unless specified, all other culture components were purchased from Miltenyi Biotech (Germany).

### SCPC Sorting Using MDDS Sorting Device

Prior to sorting, the MDDS sorter was incubated with 70% ethanol for at least 30 min for sterilization. It was then rinsed with 1× Phosphate-buffered saline (PBS) and medium. The cells, at a concentration of 0.5-1 million cells per mL, were loaded into a 50 ml syringe and injected into the device at flow rates of 5 ml/min for the low-speed mode and 9 ml/min for the high-speed mode using a syringe pump (PHD ULTRA Syringe Pumps, Harvard Apparatus, USA). To implement the recirculation strategy, a dual check valve (Quosina, USA) was used to retract the sorted cells into the input syringe by withdrawing them at a flow rate of 10 ml/min from the outlet reservoir using the syringe pump.

Cells of different size groups were loaded into a cell counting chip and had several 10x brightfield images captured for each group. The images were then analyzed via an in-house developed MATLAB code to obtain the sizes of the cells in each group.

Implantation Study with Sorted and Unsorted Cells

Female Sprague-Dawley rats (7-9 weeks, 230-250 g) were obtained from In Vivos Pte Ltd (Singapore). The rats were housed under temperature-controlled conditions, with a normal 12/12 h light/dark cycle with ad libitum access to water and food. The rats (n=16) were housed for acclimatization for 7 days and were injected with cyclosporin A (10 mg/kg, intraperitoneally, once a day) from 3 days before the surgery until perfusion. Ketamine (50 mg/kg) and xylazine (5 mg/kg) were injected intraperitoneally to anesthetize the rats. The surgical field was shaved cleaned with 70% ethanol, and betadine. Subsequently, the skin and the muscle were cut above the thoracic level, and the vertebra at levels T8-T11 were exposed. A dorsal laminectomy was performed on T9-T10. Following that, the dura was opened, and local anesthesia (lidocaine, 20 mg/mL) was applied to the spinal cord. The animals were placed on a stereotaxic system (KOPF5000 with a microinjection unit). A Hamilton syringe loaded (Model 701 N) with SCPCs (1×10⁶ cell/μL: unsorted n=4; sorted small n=4; sorted large n=4; mixed n=4), along with growth factors bFGF (10 ug/mL), VEGF (10 ug/mL), BDNF (50 ug/mL) and calpain-inhibitor (50 μM), was connected to the microinjection unit. 8 μl of SCPCs was injected (4 μl on either side of the spinal cord) at a flow rate of 0.5 μl per minute. The syringe was gently removed after two minutes to avoid backflow, and the injury area was covered with polycaprolactone film (3 mm by 3 mm with a thickness of ˜50 μm). The muscle layer was sutured with PROLENE™ 4-0 suture, followed by skin closure with wound clips. The animals were regularly monitored and were injected with buprenorphine (0.65 mg/kg) twice a day and meloxicam (1 mg/kg) once a day subcutaneously for 5 days post-injury.

### Immunohistochemical Analysis

Four weeks after in vivo cell injections, the animals were perfused with 0.9% saline, followed by 4% PFA. After perfusion, spinal cords containing the implanted cells were retrieved and post-fixed with 4% PFA for another 24 hours before transferring into 15% sucrose for 24 hours. After that, samples were transferred to 30% sucrose and stored at 4° C. until sectioning. Spinal cord samples were sectioned longitudinally at 20 μm thickness using a cryostat (Leica CM1950) and directly mounted onto glass slides. Immunofluorescent staining was performed to evaluate the cell viability and cell differentiation in vivo.

Briefly, the frozen sections were permeabilized with 0.3% TritonX-100 for 15 minutes before incubating in a blocking buffer (1% BSA diluted in 0.1PBST) for at least 1 h. Primary antibodies were then added and incubated at 4° C. overnight. The following primary antibodies were used: Oct4 (1:500) as an iPSC marker; SOX1 (1:500) as neuronal progenitor marker; SOX2 (1:200) neuronal stem cell marker; HoxB4 (1:250) as a spinal identity marker; NF200 (1:500) to identify neurofilaments; GFAP (1:1000) as an astrocytic marker; HuNu (1:200) human nuclei marker; hNCAM (1:200) as a human neural adhesion marker; NeuN (1:500) as a mature neuronal marker and Iba1 (1:1000) as an inflammatory marker. Samples were washed three times with PBS and incubated with the 488 or 555 conjugated species-specific secondary antibodies (1:1000) for 2 h at room temperature. The cell nucleus was counter-stained using DAPI (1:1000). The sections were mounted with mounting media before visualizing using Zeiss inverted confocal microscope (LSM800) and epifluorescence. FIJI/ImageJ was used to analyze the images and quantification.

### Results

In this study, iPSCs derived from Cord Lining Endothelial cells (CLECs) were differentiated into Spinal-cord Progenitor Cells (SCPCs) through a 10-day differentiation process. By profiling the size of the resultant SCPC population, we found that there was a large-sized group containing largely cells with residual pluripotent markers (i.e., Oct4), and a small-sized group with relatively fewer such cells.

A sized-based, label-free separation using an inertial microfluidic-based device was exploited to remove Oct4-positive undifferentiated cells. This device has the advantage of label-free, non-contact, and high throughput sorting (i.e., up to 3 million cells per minute and even more when stacking devices) without affecting cell viability and functions. Approximately 33 million cells can be sorted in 30 minutes into small-sized and large-sized groups. Flow cytometer analysis showed that 50% of total Oct4 positive cells in the unsorted population were sorted into the large-sized group, which contains ˜16% of the original cell number.

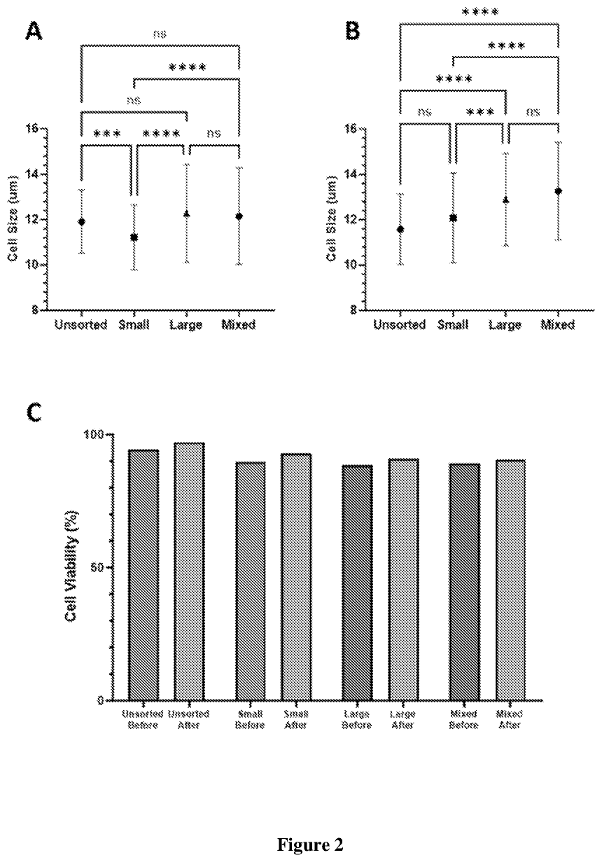

Size-based separation of differentiated and undifferentiated cells was achieved by flowing the population of cells through a double spiral microfluidic sorter. The device sorted cells into two populations through two outlets, denoted as the “small cell” outlet and the “large cell” outlet. A mixed group of cells was obtained by mixing cells from the small cell and large cell outlets at a 50-50 ratio. Quantification of cell sizes showed that the average size of cells was significantly different in the small cell and large cell groups (FIGS. 2A and 2B).

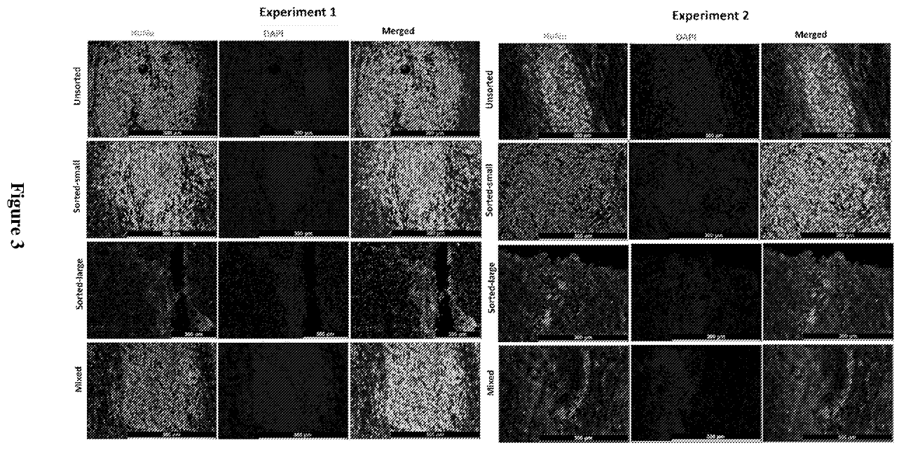

Cell viability was not heavily affected by the MDDS sorter as seen by trypan blue exclusion cell viability counts when comparing Unsorted to Sorted-small and Sorted-large cell groups (FIG. 2C). In vivo cell viability of the injected SCPCs was assessed by analyzing the intracellular location of the HuNu marker (a nuclear location identifies live SCPCs whereas a cytoplasmic location identifies dead SCPCs). Four weeks after implantation, the results showed that all the rats (n=4) that received Unsorted or Sorted-small cells had viable SCPCs (as evidenced by the dense clustering of nuclear HuNu+ cells within the implantation sites, whereas the Sorted-large cells were mostly dead (evident with cytoplasmic or no HuNu signals). The Mixed cell group had a viability of 50% (FIG. 3).

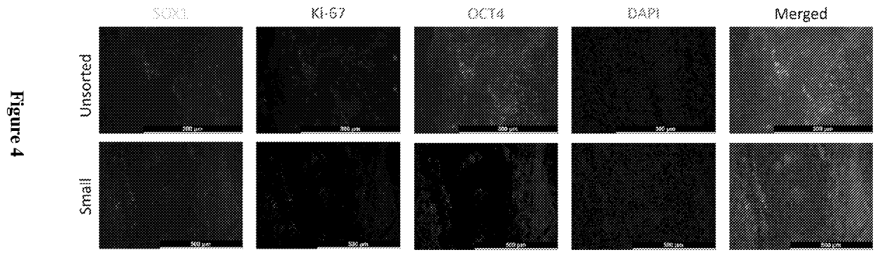

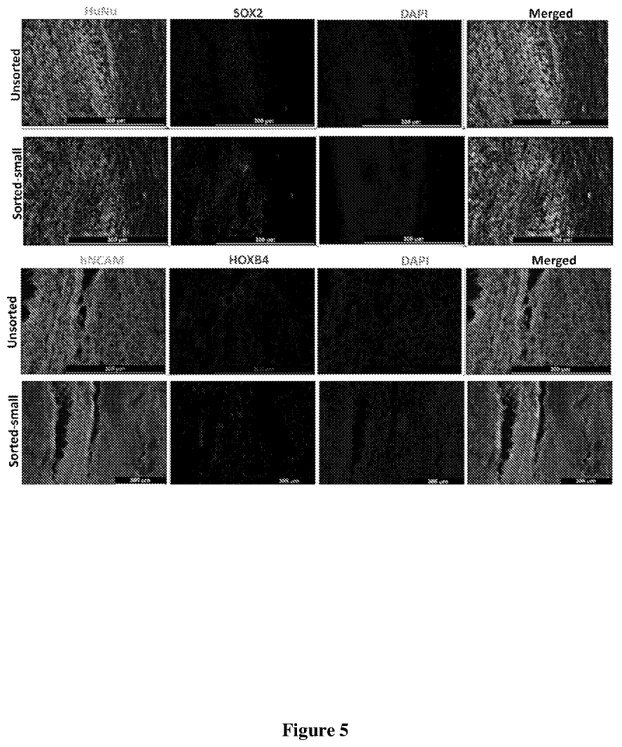

The phenotype of the injected cells of the Unsorted and Sorted-small groups was analyzed using immunostaining for pluripotency, proliferation, and progenitor markers. The results showed that the injected cells were no longer pluripotent, and most had differentiated into other cell types. Specifically, at 4 weeks post-injection, none of the cells expressed the pluripotent marker, OCT4, and only a tiny proportion of HuNu+ cells co-localized with Ki-67 and the neural progenitor marker, SOX1 (FIG. 4). Besides, the injected cells retained their spinal cord identity after transplantation, as shown by the expression of the spinal cord identity marker HoxB4 and SOX2 (FIG. 5).

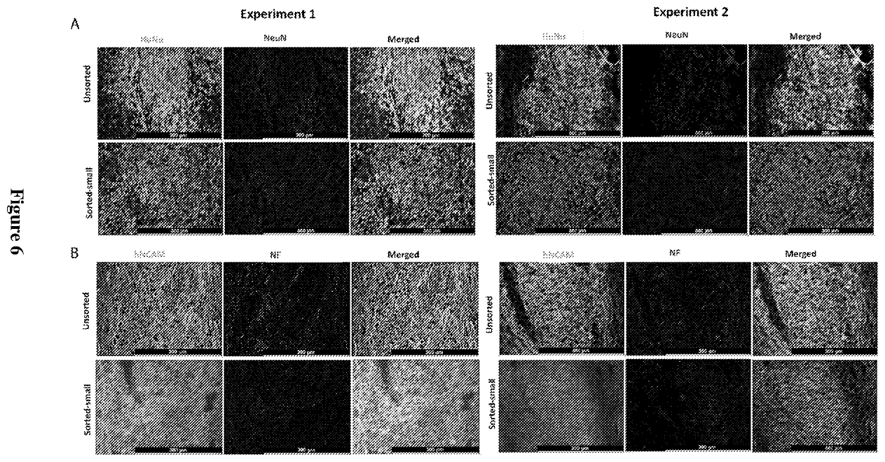

Immunostaining for pan-neuronal markers NF200 (immature) and NeuN (mature neuronal markers) showed that, while most of the viable HuNu+ cells differentiated into NeuN+, Sorted-small cells demonstrated a more extensive or equal extent of neuronal differentiation vs. unsorted cells after 4 weeks of in vivo transplantation (FIG. 5). Furthermore, co-localization of the human neural adhesion marker (hNCAM) and NF200 also validated the differentiation of SCPCs into neurons (FIG. 6).

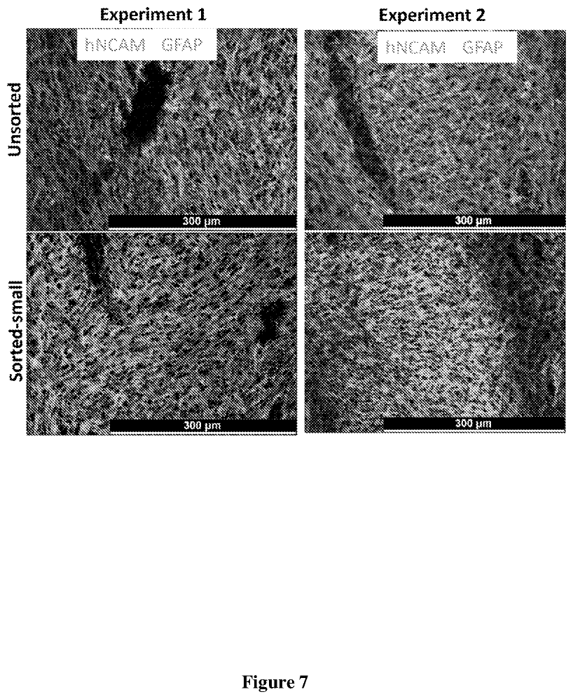

Finally, co-localization of hNCAM with GFAP showed that a sparse population of the SCPCs differentiated into astrocytes in vivo (FIG. 7). However, there was no difference between unsorted or sorted small groups within the two experimental sets.

1. A method for removing undifferentiated cells from a population of differentiated and undifferentiated cells, wherein the method comprises:
a) providing an input population of differentiated and undifferentiated cells to an inlet of a microfluidic device;
b) flowing the input population of cells through at least one curvilinear microfluidic channel coupled to the inlet, wherein the curvilinear microfluidic channel is configured to separate undifferentiated cells from the input population of cells by size; and
c) collecting a first output population of cells through one or more outlets coupled to a curvilinear microfluidic channel of the microfluidic device, wherein the first output population of cells has a lower proportion of undifferentiated cells relative to the input population of cells.

2. The method of claim 1, wherein the at least one curvilinear microfluidic channel is a spiral microfluidic channel.

3. The method of claim 1 or 2, wherein the differentiated and undifferentiated cells are mammalian cells.

4. The method of any one of claims 1 to 3, wherein the undifferentiated cells are stem cells.

5. The method of any one of claims 1 to 4, wherein the undifferentiated cells are pluripotent cells.

6. The method of claim 5, wherein the pluripotent cells are induced pluripotent stem cells (iPSCs) or human embryonic stem cells (hESCs).

7. The method of any one of claims 1 to 6, wherein the differentiated cells are spinal cord progenitor cells (SCPCs), neural progenitor cells (NPCs) and/or cells differentiated thereform.

8. The method of any one of claims 1 to 7, wherein the curvilinear channel has a non-uniform cross-sectional height.

9. The method of any one of claims 1 to 8, wherein step (b) comprises flowing the input population of cells through first and second sequentially connected and fluidically communicating curvilinear microfluidic channels, wherein the first channel is coupled to the inlet and the second channel is coupled to one or more outlets.

10. The method of claim 8, wherein the first curvilinear microfluidic channel has a smaller cross-sectional area than the second curvilinear microfluidic channel.

11. The method of claim 8 or 9, wherein the first and second curvilinear microfluidic channels are both spiral channels.

12. The method of any one of claims 1 to 10, wherein the method further comprises collecting a second output population of cells through the one or more outlets of the microfluidic device, wherein the second output population of cells has a higher proportion of undifferentiated cells relative to the input population of cells.

13. The method of any one of claims 1 to 11, wherein the method further comprises repeating steps (a) to (c) at least once, wherein the first output population of cells is provided as the input population of cells in the first and subsequent repeats.

14. The method of any one of claims 1 to 12, wherein the viability of the differentiated cells in the input population of cells and in the first output population of cells is comparable.

15. A method for purifying differentiated cells from a population of differentiated and undifferentiated cells, wherein the method comprises:
a) providing an input population of differentiated and undifferentiated cells to an inlet of a microfluidic device;
b) flowing the input population of cells through at least one curvilinear microfluidic channel coupled to the inlet, wherein the curvilinear microfluidic channel is configured to separate differentiated cells from the input population of cells by size; and
c) collecting a first output population of cells through one or more outlets coupled to the curvilinear microfluidic channel of the microfluidic device, wherein the first output population of cells has a higher proportion of differentiated cells relative to the input population of cells.

16. The method of claim 13, wherein the method comprises removing undifferentiated cells from the input population of cells.

17. A device for removing undifferentiated cells from a population of differentiated and undifferentiated cells, the device comprising:
a) an inlet for receiving an input population of differentiated and undifferentiated cells;
b) at least one curvilinear microfluidic channel coupled to the inlet, wherein the curvilinear microfluidic channel is configured to separate undifferentiated cells from the input population of cells by size; and
c) one or more outlets coupled to the curvilinear microfluidic channel for collecting an output population of cells, wherein the output population of cells has a lower proportion of undifferentiated cells relative to the input population of cells.

18. A device for purifying differentiated cells from a population of differentiated and undifferentiated cells, the device comprising:
a) an inlet for receiving an input population of differentiated and undifferentiated cells;
b) at least one curvilinear microfluidic channel coupled to the inlet, wherein the curvilinear microfluidic channel is configured to separate differentiated cells from the input population of cells by size; and
c) one or more outlets coupled to the curvilinear microfluidic channel for collecting an output population of cells, wherein the output population of cells has a higher proportion of differentiated cells relative to the input population of cells.
